(19) United States
(12) Patent Application Publication
(10) Pub. No.: US 2024/0033342 A1
(43) Pub. Date: Feb. 1, 2024

(54) COMPOSITIONS FOR TREATING GASTROINTESTINAL ADENOCARCINOMAS BY ALTERING THE TUMOR MICROENVIRONMENT

(71) Applicant: Nordic Science Group ApS, Hørsholm (DK)

(72) Inventors: Lars Otto Uttenthal, Madrid (ES); Torsten Bjørn, Kvistgård (DK)

(21) Appl. No.: 18/020,210

(22) PCT Filed: Aug. 12, 2021
(86) PCT No.: PCT/EP2021/072471

§ 371 Date: Feb. 7, 2023

(30) Foreign Application Priority Data
Aug. 17, 2020 (DK) PA 2020 70537

(51) Int. Cl. A61K 39/215; A61K 31/616; A61K 31/4178; A61K 31/341; A61P 35/00

(52) U.S. Cl. CPC A61K 39/215; A61K 31/616; A61K 31/4178; A61K 31/341; A61P 35/00; A61K 2039/53

(57) ABSTRACT

The present invention provides compositions comprising a vaccine against the SARS-CoV-2 virus for promoting an antitumor immune response in a subject with an accessible adenocarcinoma tumor who has previously been exposed to said virus by infection or vaccination, by the direct injection of the composition into the tumor.

### SEQUENCE LISTING STATEMENT

The present application contains a Sequence Listing, which is being submitted via EFS Web on even date herewith. The Sequence Listing is submitted in a file entitled “2023-02-07 Sequence Listing—ZACC259.001APC.txt,” which was created on Feb. 7, 2023, and is approximately 12,416 bytes in size, and further updated by a file entitled “2023-05-22 Sequence Listing—ZACC259.001APC.txt,” which was created on May 22, 2023, and is approximately 13,201 bytes in size. This Sequence Listing is hereby incorporated by reference.

### FIELD OF INVENTION

The present invention provides compositions designed to alter the microenvironment of gastrointestinal tumors, particularly colorectal adenocarcinomas, in such a way as to promote the killing of tumor cells by local immune mechanisms as well as to inhibit other processes that promote tumor growth and invasion, such as collagen deposition, neo-angiogenesis and the formation of micrometastases. The purpose of the invention is to promote the involution of the tumor or reduce its size and invasive activity prior to any surgical excision or ablative or other transcutaneous or endoscopic intervention directed towards the tumor that may be considered necessary. As such, it is relevant to the fields of oncology, gastroenterology and gastrointestinal surgery.

### BACKGROUND OF INVENTION

The tumor microenvironment refers to the whole biological environment surrounding tumor cells and may thus refer to both the immediate surroundings of the tumor and to conditions within a solid tumor itself. Components of this environment that are relevant to tumor growth include cellular elements such as immune cells and fibroblasts, vasculature, the extracellular matrix and signaling molecules such as cytokines, chemokines and growth factors produced by the constituent cells, which exert their actions on their immediate surroundings. In recent decades, increasing attention has been paid to these factors as targets for therapeutic procedures and agents intended to reduce tumor growth or induce tumor regression, as well as to diminish the spread of metastases from the tumor.

A principal focus of attention has been the immune cells immediately surrounding and within the tumor. Of major significance are the tumor-associated macrophages. These may be derived in different proportions from circulating monocytes or tissue-resident macrophages in different tumors, but in most cases they show functional characteristics approaching those of the M2 macrophage phenotype, exerting an overall anti-inflammatory effect and suppressing anti-tumor T cell activity. Their actions support tumor growth not only by weakening antitumor immune defenses but also by directly promoting neo-angiogenesis within and around a solid tumor. This provides for the blood supply needed for tumor growth, but also creates weak, pericyte-deficient blood vessels that are hyper-permeable and allow the escape of tumor cells to form remote metastases. An important factor in this angiogenesis is the secretion of growth factors by the macrophages, including the vascular endothelial growth factor VEGF-A and tumor necrosis factor alpha (TNFα), as well as basic fibroblast growth factor (bFGF), urokinase-type plasminogen activator (uPA) and adrenomedullin. The secretion of the interleukins IL-4, IL-6, IL-10 and IL-13 and the surface expression of programmed cell death protein 1 ligand (PD-1 ligand 1; PD-L1) by the tumor macrophages contribute to suppressing the antitumor activity of the tumor-infiltrating lymphocytes.

Among the large number investigations of circumstances and therapies that can change the tumor microenvironment in a direction that might inhibit tumor growth and metastasis and lead to tumor regression, it has recently been observed that the injection of inactivated influenza vaccine into colorectal adenocarcinomas can activate the tumor immune cell environment, characterized predominantly by suppressive regulatory T and B cell subtypes and myeloid-derived suppressor cells, by stimulating the recruitment of systemic CD8+ T cells that mediate antitumor immunity and sensitizing resistant tumors to checkpoint blockade (Newman et al 2020). The fundamental reason why the conventionally used inactivated influenza vaccine, as well as an active influenza infection, which has also been found to have an antitumor effect, should result in this antitumor immune response has not been elucidated. It is here postulated that for this type of response to occur, it is necessary that the virus and the significant viral proteins present in the vaccine should show a strong binding affinity for the tumor cells, and that the host should previously have been exposed to the virus or vaccinated against it with a vaccine that ensures that the host has T and B memory cells that can be activated by renewed exposure to viral proteins. This applies to the antitumor use of the inactivated influenza vaccine, as influenza is an endemic disease to which the vast majority, if not all, of the patients with colorectal adenocarcinomas has been exposed, often on several occasions during their lives.

### SUMMARY OF THE INVENTION

Accordingly, the present invention seeks to achieve an extremely potent antitumor effect of this strategy by exploiting the properties of the SARS-CoV-2 betacoronavirus that has recently emerged and caused a major pandemic. This new virus shows a high affinity for the cells of the gastrointestinal epithelium from which the adenocarcinomas arise. The significant viral protein for eliciting the response is known as the S or spike protein or surface glycoprotein protein of said virus, which binds via the receptor binding domain of its S1 subunit to the angiotensin converting enzyme 2 (ACE2) expressed on the host cell surface. The ACE2 thus acts as the receptor for the virus and mediates the entry of the virus into the cells. The general infectivity of the SARS-CoV-2 virus, mediated by the S protein, of which many minor variants are now known, may be several hundred-fold to thousand-fold higher to that of the commonly occurring influenza viruses. The method of the invention may be performed on accessible tumors at various sites in the body, including the lungs, and its use is not necessarily confined to the treatment of colorectal adenocarcinomas, although the latter form a major target for its application.

Therefore, the present invention comprises compositions and methods for promoting an antitumor immune response in a subject with an adenocarcinoma, as stated in the following items:
1. A composition comprising a vaccine against SARS-CoV-2 virus for use in promoting an antitumor immune response in a subject that has an adenocarcinoma tumor at a site that is accessible to injection and wherein said subject has previously been exposed to said virus by infection or vaccination, by injecting a dose of said composition directly into said tumor.
2. The composition for use according to item 1, wherein said vaccine contains whole inactivated SARS-CoV-2 virus or a recombinantly produced protein or protein fragment therefrom.
3. The composition for use according to item 1, wherein said vaccine contains a ribonucleic or deoxyribonucleic acid sequence that encodes a protein or protein fragment of said virus, and wherein said ribonucleic or deoxyribonucleic acid upon entry into a tumor cell is capable of inducing the expression of said protein or protein fragment.
4. The composition for use according to item 2, wherein said vaccine contains a recombinantly produced protein or protein fragment from the SARS-CoV-2 virus that includes the receptor binding domain of the S protein of said virus.
5. The composition for use according to item 1, wherein the dose of said vaccine that is injected into the tumor is the immunizing dose of the vaccine.
6. The composition for use according to item 4, wherein the dose of said recombinantly produced protein or protein fragment from the SARS-CoV-2 virus that is injected directly into the adenocarcinoma tumor is between 1 and 50 micrograms.
7. The composition for use according to any of the preceding items, wherein the tumor is an adenocarcinoma of the colon or rectum or a pancreatic ductal adenocarcinoma.
8. The composition for use according to any of the preceding items, wherein the tumor has previously been subjected to electroporation.
9. The composition for use according to any of the preceding items, wherein the subject is given a daily oral dose of acetylsalicylic acid 150 mg, such as between 75 and 300 mg, and/or losartan 100 mg such as between 50 and 150 mg, and/or ranitidine 150 mg such as between 75 and 300 mg, which may be given in a combined tablet, for a period of up to several weeks before and/or several weeks or months after the injection of the composition into the tumor.

As a method of treatment, these items can be stated as follows:
10. A method for promoting an antitumor immune response in a subject with an adenocarcinoma tumor at a site that is accessible to injection, said subject having previously been exposed by infection or vaccination to the SARS-CoV-2 virus, comprising injecting said tumor with a dose of a composition comprising a vaccine against said virus directly into the tumor.
11. The method according to item 10, wherein said vaccine contains whole inactivated SARS-CoV-2 virus or a recombinantly produced protein or protein fragment therefrom.
12. The method according to item 10, wherein said vaccine contains a ribonucleic or deoxyribonucleic acid sequence that encodes a protein or protein fragment of said virus.
13. The method according to item 11, wherein said vaccine contains a recombinantly produced protein or protein fragment from the SARS-CoV-2 virus that includes the receptor binding domain of the S protein of said virus.
14. The method according to item 10, wherein the dose of said vaccine that is injected into the tumor is the immunizing dose of the vaccine.
15. The method according to item 13, wherein the dose of said recombinantly produced protein or protein fragment from the SARS-CoV-2 virus that is injected directly into the adenocarcinoma tumor is between 1 and 50 micrograms.
16. The method according to any of items 10 to 15, wherein the tumor is an adenocarcinoma of the colon or rectum or a pancreatic ductal adenocarcinoma.
17. The method according to any of items 10 to 16, wherein the tumor has previously been subjected to electroporation.
18. The method according to any of claims 10 to 17, wherein the subject is given a daily oral dose of acetylsalicylic acid 150 mg, such as between 75 and 300 mg, and/or losartan 100 mg such as between 50 and 150 mg, and/or ranitidine 150 mg such as between 75 and 300 mg, which may be given in a combined tablet, for a period of up to several weeks before and/or several weeks or months after the injection of the composition into the tumor.

In practicing the invention, it may be necessary to establish that the subject has in fact been exposed to the SARS-CoV-2 virus. This can be done by testing the subject for antibodies against SARS-CoV-2 antigens, preferably antigens that include the S protein or the receptor binding domain therefrom, in view of items 4 and 13 above. If said antibodies are not detected, the subject should prior to treatment according to the methods and compositions of the invention, be vaccinated with an anti-SARS-CoV-2 vaccine approved for human use that fulfills one or more of the characteristics stated in items 2 to 4 and 11 to 13 above and retesting for the appearance of said antibodies after a period of at least 2 weeks.

The practicing of the invention may be combined with other antitumor treatments, in particular with electroporation of the tumor prior to the intra-tumor injection of the vaccine. Electroporation may be carried out at a high voltage that leads to the rapid death of tumor cells by necrosis. It may also be carried out at a lower voltage which permeabilizes the tumor cells to molecules such as cytotoxic drugs, when it is termed electrochemotherapy, and to ribonucleic acid (RNA) and deoxyribonucleic acid (DNA) molecules that encode a protein or protein fragment and is capable of inducing the expression of said protein or protein fragment upon entry into a tumor cell. As the use of an anti-SARS-CoV-2 vaccine based on RNA or DNA encoding a protein or protein fragment of the virus is envisaged in items 3 and 12 above, with the purpose that tumor cells should survive for a sufficient length of time express and present as antigens a small amount of the corresponding protein sequence, in order to promote an immune response against it, it is the latter type of electroporation that is preferred in connection with practicing the present invention. This procedure is described by Hansen et al (2020).

If the anti-SARS-CoV-2 vaccine that is used to vaccinate the subject is an RNA or DNA vaccine that exceptionally does not encode the receptor binding domain of the S protein of the virus, the vaccine that is injected into the tumor is the same vaccine as that used to immunize the subject.

The practicing of the invention may also be combined with medicinal means of promoting an antitumor microenvironment, such as the use of oral acetylsalicylic acid, with an inhibitory effect on angiogenesis, an oral angiotensin II receptor type 1 antagonist such as losartan, which affects tumor collagen synthesis and promotes the infiltration of CD8+ T cells into the tumor tissue, and an oral H₂ receptor antagonist such as ranitidine or famotidine, which have a multitude of effects to limit tumor growth and metastasis, including the promotion of tumor infiltration by lymphocytes. Such medicinal adjuvant antitumor therapy may be given in the form of a combined tablet containing acetylsalicylic acid 75 mg to 300 mg, losartan 50 mg to 150 mg and ranitidine 75 mg to 300 mg, to be taken orally once daily for a period of up to several weeks before and/or up to several weeks or months after treatment by means of the present invention. By “several weeks” is meant a period of at least 2 weeks up to 6 weeks and by “several months” is meant a period of at least 2 months up to 6 months, without implying that this limitation is a necessary aspect of the treatment. In one embodiment of the invention, the acetylsalicylic acid is administered in a dose of 150 mg, the losartan dose is 100 mg and the ranitidine dose is 150 mg.

In the following detailed description of the invention, details of the scope of the invention and its practical performance will be given.

### DETAILED DESCRIPTION OF THE INVENTION

The essential feature of the present invention is the use of an anti-SARS-CoV-2 vaccine as a means of inducing an antitumor immune response in a subject with an adenocarcinoma, wherein said subject has previously been exposed to the SARS-CoV-2 virus by infection or by vaccination against it, by the direct intra-tumor injection of a composition containing said vaccine.

The suitability of the subject for the treatment is demonstrated by the detection of antibodies against SARS-CoV-2 antigens, preferably antigens that include the S protein or the receptor binding domain therefrom, in a serum, plasma or whole blood sample taken from the subject. If no such antibodies are detected, the subject is offered vaccination with an anti-SARS-CoV-2 vaccine approved for human use that fulfills one or more of the following characteristics:
a) The vaccine contains whole inactivated SARS-CoV-2 virus or a recombinantly produced protein or protein fragment therefrom.
b) The vaccine contains an RNA or DNA sequence that encodes a protein or protein fragment of the SARS-CoV-2 virus, and which RNA or DNA is capable of inducing the expression of said protein or protein fragment upon entering the tumor cell.
c) The vaccine contains a recombinantly produced protein or protein fragment from the SARS-CoV-2 virus that includes the receptor binding domain of the S protein of the virus.

Treatment is then deferred until the antibodies described above can be detected.

The treatment consists of injecting a composition containing a vaccine with one or more of the characteristics stated in a), b) and c) above, dissolved or dispersed in an appropriate aqueous medium, directly into the tumor. In some embodiments, the protein or protein fragment mentioned in c) above and for use in the compositions and methods of the invention is between 10 and 1273 amino acids long and the sequence is at least 75%, such as at least 80%, such as at least 90% identical to that of the comparable region of SEQ ID NO:1, or the protein fragment is between 10 and 194 amino acids long and is at least 75%, such as at least 80%, such as at least 90% identical to that of the comparable region of SEQ ID NO:2.

For the purpose of the present invention, it is not considered necessary that any nucleotide-based vaccine should have the sequence coding for the viral protein incorporated into a virus, such as an adenovirus, as a carrier that is capable or limitedly capable of replicating within the human body or capable of producing further viral protein. Indeed, such a measure may introduce the chance of side effects due to infection by the carrier virus, or reactions to proteins of the carrier virus, or to the chance of inactivation of the vaccine by pre-existing antibodies against the carrier virus that the subject may have. It is not a purpose of the invention that any SARS-CoV-2 antigen should be expressed at non-tumor sites remote from the tumor to be treated.

The injection may be preceded by electroporation of the tumor by means that are well known to the skilled person, and may be followed by daily oral medication with a combination of drugs comprising acetyl salicylic acid, an angiotensin II receptor type 1 antagonist, and an H₂ receptor antagonist.

### Active Ingredients of Compositions of the Invention

The essential active ingredient of a composition of the invention is an anti-SARS-CoV-2 vaccine suitable for human use. These include the following active ingredients:
1) Whole inactivated SARS-CoV-2 virus, e.g. inactivated by treatment with p-propiolactone or formaldehyde in a manner similar to the production of standard inactivated seasonal influenza vaccine.

For the purpose of promoting an antitumor immune response, it is not expected that the chemical modification of the active ingredient that will be produced by the inactivation of whole virus by the method described will destroy its capacity to induce such a response. A candidate vaccine of this type has been reported by Gao et al (2020).
2) Recombinantly produced SARS-CoV-2 S protein (also known as the spike protein or surface glycoprotein) or a recombinantly produced fragment of SARS-CoV-2 S protein that includes the receptor binding domain of the S1 subunit of that protein.

The production of the simpler forms of this active ingredient is described in more detail below.
3) An RNA or DNA sequence that encodes a protein or protein fragment of the SARS-CoV-2 virus.

This type of active ingredient will usually be presented together with one or more adjuvants and permeants to facilitate the penetration of these rather large molecules into the cells of the vaccinated host, so that the protein material that it encodes will be expressed and presented as an antigen for provoking an immune response against them, and hence against the virus. An RNA sequence of this type may be incorporated into a virus, such as an adenovirus, that is capable of replicating within the immunized subject and infect host cells, such that they express the encoded viral protein. This procedure is not considered necessary or advantageous to the practice of the present invention and may indeed have negative consequences in the form of side effects or the inactivation of the vaccine by pre-existing antibodies, but does not preclude its use in the invention.

If the RNA or DNA sequence in the vaccine used to immunize the subject exceptionally does not encode the receptor binding domain of the S protein of the virus, the active ingredient that is injected into the tumor is the same vaccine as that used to immunize the patient.

### Production of Recombinantly Expressed SARS-CoV-2 S Protein or a Fragment Thereof that Includes the Receptor (ACE2) Binding Domain

In one embodiment of the invention, the active ingredient present in the composition for direct injection into the adenocarcinoma tumor to be treated is recombinantly produced S protein of the SARS-CoV-2 betacoronavirus [SEQ ID NO:1], or any fragment of said protein that includes the receptor (ACE2) binding domain [SEQ ID NO:2] of its S1 subunit. The protein sequences have been reported by Wu et al (2020).

The protein or protein fragment can be recombinantly produced by expression in host cells such as prokaryotic cells, yeast cells, insect cells, mammalian cells or in cell-free systems.

In one embodiment of the invention, the S protein or fragment thereof is produced recombinantly by host cells. Thus, in one aspect of the present invention, the protein or protein fragment is produced by host cells comprising a first nucleic acid sequence encoding the protein or protein fragment, operably associated with a second nucleic acid sequence capable of directing expression in said host cells. The second nucleic acid sequence may thus comprise or even consist of a promoter that will direct the expression of protein of interest in said cells. A skilled person will be readily capable of identifying useful second nucleic acid sequence for use in a given host cell.

The process of producing the recombinant protein in general comprises the steps of:
providing a host cell;
preparing a gene expression construct comprising a first nucleic acid sequence encoding the S protein or fragment thereof to be expressed, operably linked to a second nucleic acid sequence capable of directing the expression of said protein or fragment in the host cell;
transforming the host cell with the construct
cultivating the host cell, thereby obtaining expression of the protein or fragment thereof.

The recombinant S protein or fragment thereof thus produced may be isolated by any conventional method known to the skilled person, who will be able to identify suitable protein isolation steps for purifying the product.

In one embodiment of the invention, the recombinantly produced protein or fragment of interest is excreted by the host cells. In this case, the process of producing the recombinant protein or protein fragment may comprise the previously stated steps, in which cultivating the transformed host cell results in the expression of the protein or protein fragment and its secretion into the culture medium, thereby obtaining culture medium containing the product of interest.

The composition comprising the S protein or protein fragment thereof may thus in this embodiment of the invention be the culture medium or a composition prepared from the culture medium.

In an embodiment of the invention, the S protein or fragment thereof is recombinantly produced in vitro in host cells and isolated from cell lysate, cell extract or from tissue culture supernatant. In a more preferred embodiment, the S protein or fragment thereof is produced by host cells that are modified in such a way that they express the relevant protein or protein fragment. In an even more preferred embodiment of the invention, said host cells are transformed to produce and excrete the relevant protein or protein fragment.

For the purpose of the present invention, i.e. to induce an antitumor immune response, it is not believed that it is necessary for the active ingredient to be glycosylated. Therefore it may be produced in adequate form by expression in cells that do not glycosylate the expressed proteins, such as in Escherichia coli.

The amino-acid sequences of the proteins of the SARS-CoV-2 virus are not invariable and slight differences in amino-acid sequence between the proteins of different clinical isolates of the virus are being reported on a daily basis. This applies also to the S protein and its S1 subunit. This protein and its receptor binding domain may therefore exist as a considerable number of variants which are homologous to the index protein, the S protein of the virus as first isolated and sequenced in China [SEQ ID NO:1] and which share its role in the infectivity and pathogenicity of the virus, but which differ from it in that one or more amino-acid residues within the its sequence are substituted by other amino-acid residues. These substitutions may be regarded as “conservative” when an amino-acid residue is replaced by a different amino-acid residue with broadly similar properties, and “non-conservative” when an amino-acid residue is replaced by one of a different type. Broadly speaking, fewer non-conservative substitutions will be possible without altering the biological activity of the protein.

For the purposes of the present invention any such variant of the index protein of the receptor binding domain thereof that retains the antigenicity of the index protein or its receptor domain, whether it is naturally occurring or has been deliberately produced when the recombinant protein is expressed as described above, by modifying the nucleotide sequence encoding the protein, is suitable for practicing the invention.

A variant within the scope of the present invention may therefore be a protein that exhibits at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the comparable part of SEQ ID NO:1.

Sequence identity can be calculated using a number of well-known algorithms and applying a number of different gap penalties. Any sequence alignment algorithm, such as but not limited to FASTA, BLAST, or GETSEQ, may be used for searching homologues and calculating sequence identity. Moreover, when appropriate, any commonly known substitution matrix, such as but not limited to PAM, BLOSSUM or PSSM matrices, may be applied with the search algorithm. For example, a PSSM (position specific scoring matrix) may be applied via the PSI-BLAST program. Moreover, sequence alignments may be performed using a range of penalties for gap-opening and extension. For example, the BLAST algorithm may be used with a gap-opening penalty in the range 5-12, and a gap-extension penalty in the range 1-2.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino-acid substitution as defined above.

### Indications for Use of a Composition of the Invention

The Indications for the intra-tumor injection of a dose of a composition of the invention into an accessible adenocarcinoma are as the treating clinician determines.

### Formulations

The active ingredient of the invention is formulated for injection directly into the tumor in an aqueous medium. It may be dissolved in a pharmaceutically acceptable and approved isotonic saline solution such as sodium phosphate-buffered isotonic sodium chloride solution to a concentration of active ingredient that allows an immunizing dose to be injected directly into the tumor. In the embodiment of the invention in which the active ingredient is a recombinantly produced protein or protein fragment from the SARS-CoV-2 virus that includes the receptor binding domain of the S protein of said virus, the concentration of the active ingredient in the formulation may range from 0.05 mg/mL to 0.2 mg/mL, preferably 0.1 mg/mL. The protein solution may also optionally contain octoxynol-10 (TRITON X-100) up to a concentration of 0.2 mg/mL and/or polysorbate 80 (Tween 80) up to a concentration of 1 mg/mL as solubilizers and stabilizers, and α-tocopheryl hydrogen succinate up to a concentration of 0.25 mg/mL as an antioxidant preservative, as well as an approved concentration of an approved antibacterial preservative.

### Administration

Administration of the chosen dose of the composition is by direct injection into the substance of the tumor in a volume ranging from 10 microliters to 0.5 mL as determined by the size and conditions of the tumor. The injection is usually performed via an endoscope to get access to and to visualize the tumor.

### Dosage

The dose of an anti-SARS-CoV-2 vaccine to be injected directly into the tumor is the standard dose for immunizing an adult human subject. In the embodiment of the invention in which the active ingredient is a recombinant protein or protein fragment, the volume of the composition to be injected is chosen so that a dose of active ingredient ranging from 1 microgram to 50 microgram, preferably 15 microgram to 30 microgram, is given into the tumor. The dose is not intended to be repeated, but may be repeated after an interval of 4 weeks or more.

### Combination with Other Antitumor Interventions

Electroporation or electrochemotherapy: The provocation of an antitumor immune response by intra-tumor injection of a composition of the invention is expected to be particularly effective if the tumor has been subjected to electroporation by standard techniques prior to the injection. If the procedure in combined with the administration of anticancer chemotherapeutic agents such as bleomycin or cisplatin, it is called electrochemotherapy. Electroporation may be carried out at a high voltage that leads to the rapid death of tumor cells by necrosis. It may also be carried out at a lower voltage which permeabilizes the tumor cells to molecules such as cytotoxic drugs and RNA or DNA molecules without immediately killing the cells. When an anti-SARS-CoV-2 vaccine based on RNA or DNA encoding a protein or protein fragment of the virus is used in the present invention, the purpose of electroporation is that it should facilitate the uptake of the RNA or DNA into the tumor cells, which should survive for a sufficient length of time express and present as antigens a small amount of the corresponding protein sequence, in order to promote the antitumor immune response. Therefore it is the lower voltage type of electroporation that is preferred in connection with practicing the present invention. This procedure is described by Hansen et al (2020). DNA and RNA molecules for use in such electroporation mediated treatment would be of a nature that allow for expression of the encoded protein in a tumor cell. In case of the DNA sequences, this would include sequences directing transcription of the relevant sequences. Making such DNA constructs is well known in the art. RNA sequences for use in the present invention, such as for electroporation into tumor cells and subsequent expression of encoded protein, would include relevant sequences for mediating the initiation of the translation process. Methods for making such RNA sequences capable of being translated into protein upon entering a cell is well known in the art.

Medication to promote an antitumor microenvironment: The provocation of an antitumor immune response by intra-tumor injection of a composition of the invention can be consolidated by combining it with medicinal means of promoting an antitumor microenvironment, such as the use of oral acetylsalicylic acid, with an inhibitory effect on angiogenesis, an oral angiotensin II receptor type 1 antagonist such as losartan, which affects tumor collagen synthesis and promotes the infiltration of CD8+ T cells into the tumor tissue, and an oral H2 receptor antagonist such as ranitidine or famotidine, which have a multitude of effects to limit tumor growth and metastasis, including the promotion of tumor infiltration by lymphocytes. Such medicinal adjuvant antitumor therapy may be given by administering the individual agents separately, or may also be in the form of a combined tablet containing acetylsalicylic acid 75 mg to 300 mg, losartan 50 mg to 150 mg and ranitidine 75 mg to 300 mg, to be taken orally once daily. In one embodiment of the invention, the acetylsalicylic acid is administered in a dose of 150 mg, the losartan dose is 100 mg and the ranitidine dose is 150 mg. The period for such medical adjuvant treatment may start up to several weeks before the injection and may be continued as soon as the patient is able to take oral medication after the intra-tumor injection has been performed, and may last for a further period of up to several weeks or months at the clinician's discretion.

### Examples

### SEQ ID NO:1

### SARS-CoV-2 Spike or S Protein, or Surface Glycoprotein

Amino-acid sequence denoted by the internationally accepted single-letter amino-acid code, from residue 1 to residue 1273:

MFVFLVLLPL VSSQCVNLTT RTQLPPAYTN SFTRGVYYPD
KVFRSSVLHS TQDLFLPFFS NVTWFHAIHV SGTNGTKRFD
NPVLPENDGV YFASTEKSNI IRGWIFGTTL DSKTQSLLIV
NNATNVVIKV CEFQFCNDPF LGVYYHKNNK SWMESEFRVY
SSANNCTFEY VSQPFLMDLE GKQGNFKNLR EFVFKNIDGY
FKIYSKHTPI NLVRDLPQGF SALEPLVDLP IGINITRFQT
LLALHRSYLT PGDSSSGWTA GAAAYYVGYL QPRTFLLKYN
ENGTITDAVD CALDPLSETK CTLKSFTVEK GIYQTSNERV
QPTESIVRFP NITNLCPFGE VFNATRFASV YAWNRKRISN
CVADYSVLYN SASFSTFKCY GVSPTKLNDL CFTNVYADSF
VIRGDEVRQI APGQTGKIAD YNYKLPDDFT GCVIAWNSNN
LDSKVGGNYN YLYRLFRKSN LKPFERDIST EIYQAGSTPC
NGVEGENCYF PLQSYGFQPT NGVGYQPYRV VVLSFELLHA
PATVCGPKKS TNLVKNKCVN FNFNGLTGTG VLTESNKKFL
PFQQFGRDIA DTTDAVRDPQ TLEILDITPC SFGGVSVITP
GTNTSNQVAV LYQDVNCTEV PVAIHADQLT PTWRVYSTGS
NVFQTRAGCL IGAEHVNNSY ECDIPIGAGI CASYQTQTNS
PRRARSVASQ SIIAYTMSLG AENSVAYSNN SIAIPTNFTI
SVTTEILPVS MTKTSVDCTM YICGDSTECS NLLLQYGSFC
TQLNRALTGI AVEQDKNTQE VFAQVKQIYK TPPIKDFGGF
NFSQILPDPS KPSKRSFIED LLFNKVTLAD AGFIKQYGDC
LGDIAARDLI CAQKFNGLTV LPPLLTDEMI AQYTSALLAG
TITSGWTFGA GAALQIPFAM QMAYRFNGIG VTQNVLYENQ
KLIANQFNSA IGKIQDSLSS TASALGKLQD VVNQNAQALN
TLVKQLSSNF GAISSVLNDI LSRLDKVEAE VQIDRLITGR
LQSLQTYVTQ QLIRAAEIRA SANLAATKMS ECVLGQSKRV
DFCGKGYHLM SFPQSAPHGV VFLHVTYVPA QEKNFTTAPA
ICHDGKAHFP REGVFVSNGT HWFVTQRNFY EPQIITTDNT
FVSGNCDVVI GIVNNTVYDP LQPELDSFKE ELDKYFKNHT
SPDVDLGDIS GINASVVNIQ KEIDRLNEVA KNLNESLIDL
QELGKYEQYI KWPWYIWLGF IAGLIAIVMV TIMLCCMTSC
CSCLKGCCSC GSCCKFDEDD SEPVLKGVKL HYT

### SEQ ID NO:2

### Receptor Binding Domain of the S1 subunit of the SARS-CoV-2 Spike or S Protein or Surface Glycoprotein

Amino-acid sequence denoted by the internationally accepted single-letter amino-acid code, from residue 331 to residue 524 of the spike protein:

NITNLCPFGE VFNATRFASV
YAWNRKRISN CVADYSVLYN SASFSTFKCY GVSPTKLNDL
CFTNVYADSF VIRGDEVRQI APGQTGKIAD YNYKLPDDFT
GCVIAWNSNN LDSKVGGNYN YLYRLFRKSN LKPFERDIST
EIYQAGSTPC NGVEGENCYF PLQSYGFQPT
NGVGYQPYRV VVLSFELLHA PATV

### REFERENCES

Gao Q et al (2020) Development of an inactivated vaccine candidate for SARS-CoV-2. Science 369:77-81.
Hansen H F et al (2020) Electrochemotherapy for colorectal cancer using endoscopic electroporation: a phase 1 clinical study. Endosc Int Open 08:E124-E132.
Newman J H et al (2020) Intratumoral injection of the seasonal flu shot converts immunologically cold tumors to hot and serves as an immunotherapy for cancer. Proc Natl Aced Sci USA 117:1119-1128.
Wu F et al (2020) A new coronavirus associated with human respiratory disease in China. Nature 579:265-269.

1. A method for promoting an antitumor immune response in a subject that has an adenocarcinoma tumor comprising:
selecting a subject that has an adenocarcinoma tumor, wherein said subject has also been exposed to SARS-CoV-2 virus or a SARS-CoV-2 vaccination; and
administering to said selected subject by injection directly into said adenocarcinoma tumor a composition comprising a SARS-CoV-2 virus vaccine.

2-20. (canceled)

21. A method for promoting an antitumor immune response in a subject with an adenocarcinoma tumor at a site that is accessible to injection, said subject having previously been exposed by infection or vaccination to the SARS-CoV-2 virus, comprising injecting said tumor with an effective dose of a composition comprising a vaccine against said virus directly into the tumor.

22. The method according to claim 21, wherein said vaccine contains whole inactivated SARS-CoV-2 virus or a recombinantly produced protein or protein fragment therefrom.

23. The method according to claim 21, wherein said vaccine contains a ribonucleic or deoxyribonucleic acid sequence that encodes a protein or protein fragment of said virus, and wherein said ribonucleic or deoxyribonucleic acid upon entry into a tumor cell is capable of inducing the expression of said protein or protein fragment.

24. The method according to claim 22, wherein said vaccine contains a recombinantly produced protein or protein fragment from the SARS-CoV-2 virus that includes the receptor binding domain of the S protein of said virus.

25. The method according to claim 21, wherein the dose of said vaccine that is injected into the tumor is an immunizing dose of the vaccine.

26. The method according to claim 24, wherein the dose of said recombinantly produced protein or protein fragment from the SARS-CoV-2 virus that is injected directly into the adenocarcinoma tumor is between 1 and 50 micrograms.

27. The method according to claim 21, wherein the tumor is an adenocarcinoma of the colon or rectum or a pancreatic duct adenocarcinoma.

28. The method according to claim 21, wherein the tumor has previously been subjected to electroporation.

29. The method according to claim 21, wherein the subject is given an effective daily oral dose of acetylsalicylic acid, and/or losartan, and/or ranitidine, which may be given in a combined tablet, for a period of up to several weeks before and/or several weeks or months after the injection of the composition into the tumor.

30. The method according to claim 29, wherein the effective daily oral dose of acetylsalicylic acid is in the range of 75 mg to 300 mg, and/or that of losartan is in the range of 50 mg to 150 mg, and/or that of ranitidine is in the range of 75 mg to 300 mg.


SEQUENCE LISTING (2 sequences)

SEQ ID NO: 1 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 194 aa, Unknown
  SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV